(19) United States
(12) Patent Application Publication
(10) Pub. No.: US 2017/0182311 A1
(43) Pub. Date: Jun. 29, 2017

(54) PROCESS FOR ELECTRICALLY CONTACTING A COATED LEAD WITH A LAYER

(71) Applicant: Heraeus Deutschland GmbH & Co. KG, Hanau (DE)

(72) Inventors: Oliver KEITEL, Aschaffenburg (DE); Jens TROETZSCHEL, Ronneburg (DE); Stefan SCHIBLI, Frankfurt (DE)

(73) Assignee: Heraeus Deutschland GmbH & Co. KG, Hanau (DE)

(21) Appl. No.: 15/389,106

(22) Filed: Dec. 22, 2016

(30) Foreign Application Priority Data
Dec. 23, 2015 (EP) 15202412.1

(51) Int. Cl. A61N 1/05; A61N 1/362; A61B 5/00

(52) U.S. Cl. CPC A61N 1/05; A61B 5/6846; A61N 1/0551; A61N 1/362

(57) ABSTRACT

One aspect relates generally to a process for electrically contacting a coated lead. One aspect relates to a process for electrically contacting a coated lead including providing a coated lead comprising an electrically conductive core and an electrically insulating coating. A via hole is provided in the electrically insulating coating in order to expose a section of the electrically conductive core. A first electrically conductive material is applied to the coated lead such that it contacts at least a part of the exposed section of the electrically conductive core via the via hole. Further electrically conductive material is applied to the coated lead such that it contacts at least a part of the first conductive material.

### CROSS-REFERENCE TO RELATED APPLICATIONS

This Utility Patent Application claims priority to European Patent Application No. EP 15202412.1, filed on Dec. 23, 2015, which is incorporated herein by reference.

### BACKGROUND

One aspect relates generally to a process for electrically contacting a coated lead. More specifically, one aspect relates to a process, an electrically contacted lead and a medical device comprising an electrically contacted lead.

Making electrical contact with an electrically conductive lead can often represent a significant challenge. In order to take advantage of beneficial properties of an electrical lead, such as good electrical conductivity and good durability, the contact to the lead itself must also display similar beneficial properties. Such considerations are particularly pertinent for example when electrically contacting leads in medical devices. In devices which are introduced into the body, it is desirable to employ thin leads, which can represent a challenge to electrically contact due their size. Furthermore, a very high value is placed on reliability in medical devices such as Cardiac Pacemakers, Implantable Cardioverter Defibrillation Devices and Cardiac Resynchronisation Devices, especially in terms of resistance to physical fatigue. Invasive surgery is required to implant medical devices into the body or remove or replace parts, and it is highly desirable for the individual components of the device to have a long working life in order to reduce the requirement for surgical intervention. Furthermore, it is desirable for the working life to have a low variance. One component of a medical device which is exposed to a particularly high amount of stress during normal operation is the lead and the electrical connections thereto.

Document U.S. Pat. No. 7,364,479 B1 discloses a method for contacting a lead by crimping. Direct crimping can result in a contact which is lost over time due to physical movement. Furthermore, the direct crimping method is not suitable for multi-core leads since contact would be made to the cores indiscriminately.

Document US 2013/0338745 A1 discloses a method for contacting individual conductive cores of a cable which includes multiple conductive cores. The individually coated conductive cores are fed through the lumens of a ring, and electrical contact is made with the individual cores by piercing. This method suffers at least from the disadvantages associated with moveable parts.

For these and other reasons, a need exists for the present invention.

### SUMMARY

One embodiment is generally based on the object of overcoming at least one of the problems encountered in the state of the art in relation to electrical contacts with leads.

More specifically, one embodiment is based on the object of providing a process for electrically contacting a lead, which provides for one or more selected from the following group of advantages: an improved electrical contact, an improved durability and a more flexible process.

One object of one embodiment is to provide a process which is suitable for electrically contacting a thin lead, in one embodiment while providing for one or more selected from the following group of advantages: an improved electrical contact, an improved durability and a more flexible process.

Another object of one embodiment is to provide a process which is suitable for electrically contacting a lead for use in a medical device, in one embodiment a thin lead. In one embodiment, it is preferred for electrically contacting a lead in a bio-compatible device, in one embodiment while providing for one or more selected from the following group of advantages: an improved electrical contact, an improved durability and a more flexible process. One aspect of this object is to provide a process for connecting a lead which fulfils the requirements laid out in the “Guidance for the Submission of Research and Marketing Applications for Permanent Pacemaker Leads and for Pacemaker Lead Adaptor 510(k) Submissions” issued by the “Center for Devices and Radiological Health” of the US Food and Drug Administration. A further object is provide a method for electrically contacting a multi-core lead, in particular for discriminately contacting a single core thereof. This has long been attempted and there are various approaches provided in the art. Some of these have been discussed above. There remains, however, a need for further improvement of such electrical contacts and methods for making them.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

One aspect is now further illustrated using figures which are not to be considered as limiting the scope. In brief, the figures illustrate the following:

FIGS. 1a to 1d illustrate schematically the process for electrically contacting a coated lead.

FIG. 2 illustrates schematically a process according to one embodiment for contacting a coated lead.

FIG. 3 illustrates schematically a pacemaker comprising a lead electrically connected according to one embodiment.

FIG. 4 illustrates schematically a lead with 3 electrically conductive cores, contacted six times according to one embodiment.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

A contribution to achieving at least one of the above described objects is made by the subject matter of the category forming claims. A further contribution is made by the subject matter of the dependent claims, which represent specific embodiments.

A contribution to achieving at least one of the above described objects is made by the following embodiments.
1. A process for electrically contacting a coated lead comprising the following steps:
   a. Providing a coated lead comprising an electrically conductive core and an electrically insulating coating;
   b. Providing a via hole in the electrically insulating coating in order to expose a section of the electrically conductive core;
   c. Applying a first electrically conductive material to the coated lead such that it contacts at least a part of the exposed section of the electrically conductive core via the via hole;
   d. Applying a further electrically conductive material to the coated lead such that it contacts at least a part of the first conductive material.
2. The process according to embodiment 1, wherein the first electrically conductive material includes a polymer, in one embodiment an electrically conductive polymer having a specific conductivity greater than about 10 S/cm, in one embodiment greater than about 100 S/cm, and in one embodiment greater than about 500 S/cm. In some cases the specific conductivity of the conductive polymer can be as high as about 2000 S/cm or less.
3. The process according to embodiment 1 or 2, wherein the first electrically conductive material is one or more selected from the group consisting of: an electrically conductive polymer, a composite comprising an electrically non-conductive polymer and an electrically conductive particle, and a composite comprising an electrically conductive polymer and an electrically conductive particle. Electrically conductive particles in this context are electrically conductive carbon particles or metal particles or both.
4. The process according to any of the preceding embodiments, wherein the first electrically conductive material includes a polymer having two or more conjugated double bonds.
5. The process according to any of the preceding embodiments, wherein the first electrically conductive material is applied as a layer with thickness less than about 0.3 mm, in one embodiment less than about 0.2 mm, in one embodiment less than about 0.1 mm, and in one embodiment less than about 0.05 mm. In some cases the thickness of the layer of the first electrically conductive material can be as little as about 0.01 mm or more.
6. The process according to any of the preceding embodiments, wherein the first electrically conductive material has a greater elasticity than the second electrically conductive material. In one aspect of this embodiment, the first electrically conductive material has a greater Young's modulus of elasticity than the second electrically conductive material, in one embodiment being at least about twice, in one embodiment at least about three times, and in one embodiment still at least about 4 times its value.
7. The process according to any of the preceding embodiments, wherein the first electrically conductive material is applied by one or more selected from the group consisting of: dipping, printing, spraying, injecting, painting, dropping, stamping, spilling, wrapping and laying.
8. The process according to any of the preceding embodiments, wherein the second electrically conductive material is applied by solid deformation.
9. The process according to any of the preceding embodiments, wherein the application of the second electrically conductive material in step d. comprises one or more selected from the group consisting of: crimping, notching, bending, twisting, screwing and press fitting, in one embodiment crimping.
10. The process according to any of the preceding embodiments, wherein the second electrically conductive material is a metal.
11. The process according to any of the preceding embodiments, wherein the lead has a diameter less than about 2 mm, in one embodiment less than about 1.5 mm, in one embodiment less than about 1 mm, and in one embodiment less than about 0.7 mm. In some cases, the thickness of the lead is as little as 0.1 mm or more.
12. The process according to any of the preceding embodiments, wherein the electrically conductive core is a metal.
13. The process according to any of the preceding embodiments, wherein the electrically insulating coating is a polymer.
14. The process according to any of the preceding embodiments, wherein the lead includes 2 or more electrically conductive cores, in one embodiment 3 or more, and in one embodiment 5 or more.
15. The process according to embodiment 14, wherein 2 or more of the electrically conductive cores are electrically insulated from each other by an insulating material.
16. The process according to any of the preceding embodiments, wherein the via hole exposes a single conductive core.
17. The process according to any of the preceding embodiments, wherein the section of the electrically conductive core exposed by the via hole has a surface area in the range from about 0.001 to about 0.1 mm², in one embodiment in the range from about 0.005 to about 0.08 mm², and in one embodiment in the range from about 0.008 to about 0.04 mm².
18. The process according to any of the preceding embodiments, wherein the provision of the via hole in step b. is performed with a laser.
19. The process according to any of the preceding embodiments, wherein the first electrically conductive material has a mass in the range from about 0.01 to about 0.5 μg, in one embodiment in the range from about 0.02 to about 0.3 μg, and in one embodiment in the range from about 0.03 to about 0.2 μg.

20. An electrically contacted lead obtainable by a method according to any of the preceding embodiments.
21. An electrically contacted lead comprising the following:
   a. an electrically conductive core
   b. an electrically insulating coating which coats the electrically conductive core;
   c. a via hole in the electrically insulating coating which exposes a section of the electrically conductive core;
   d. a first electrically conductive material in the via hole which contacts at least a part of the exposed section of the electrically conductive core;
   e. a further electrically conductive material which contacts at least a part of the first conductive material.

The features introduced in relation to the above process embodiments apply mutatis mutandis as aspects of this embodiment.
22. A medical device comprising a contacted lead according to embodiment 20 or 21.
23. A medical device according to embodiment 22, selected from the group consisting of: a pacemaker, a neuro-stimulator, a measuring device and a defibrillator.

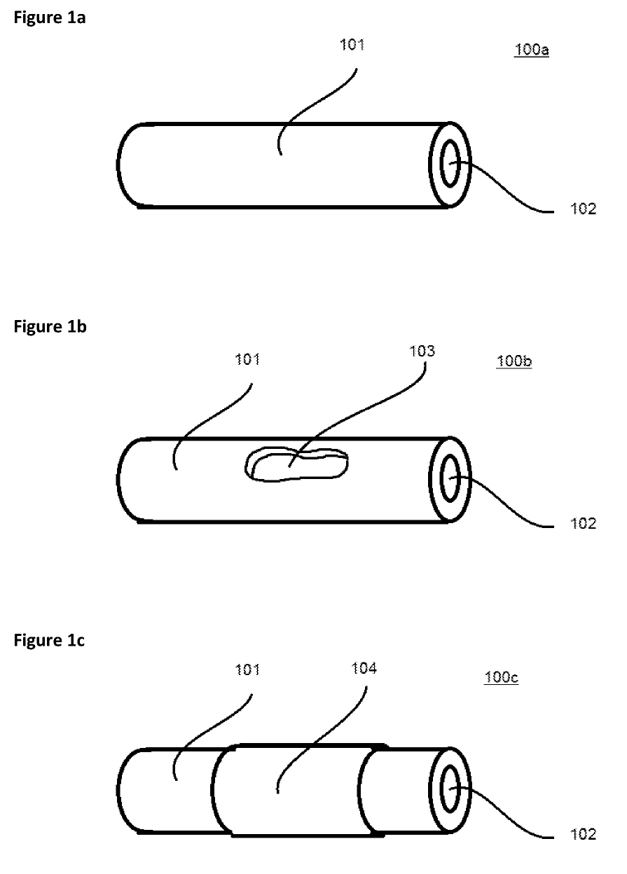

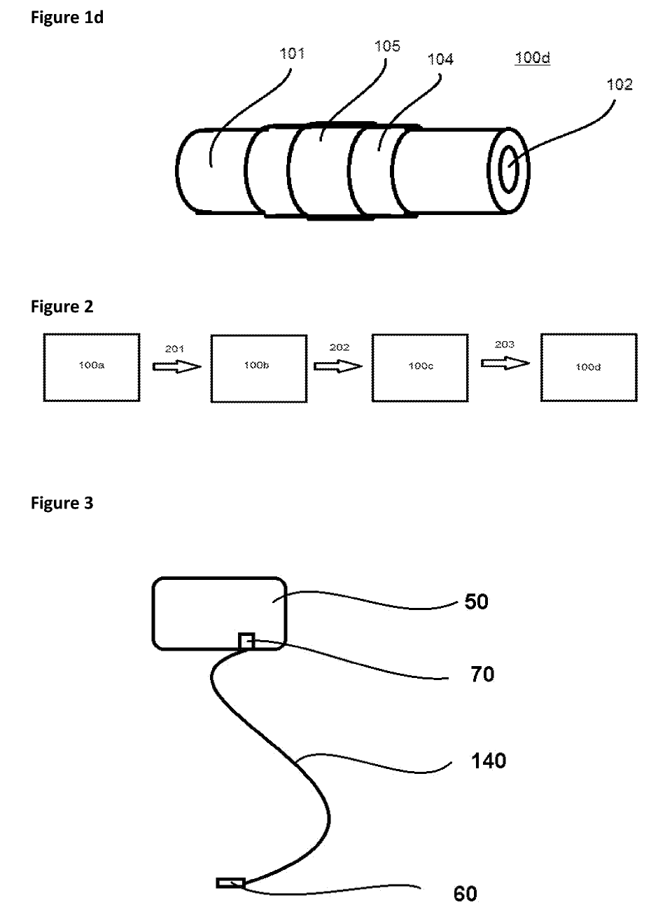

FIG. 1a illustrates a coated lead 100a according to one embodiment. The coated lead 100a includes an electrically conductive core 102 of electrically conductive material coated with an electrically insulating coating 101 of electrically insulating material. In this case, the electrically conductive material is a rhodium/platinum alloy (20% rhodium and 80% platinum, by mass) and the electrically conductive core has a diameter of 0.5 mm. In this case, the electrically insulating coating is 20 μm thick and the electrically insulating coating is of polyurethane.

FIG. 1b illustrates 100b a coated lead with via hole 103. The coated lead with via hole 100b was obtained from the coated lead 100a by laser ablation. The via hole 103 extends through the electrically insulating coating 101 to expose a section of the surface of the electrically conductive core with a cross sectional area of 0.02 mm².

FIG. 1c illustrates 100c a coated lead with a via hole 103 and a layer of electrically conductive material 104, in this case a 25 μm thick layer of PEDOT:PSS, applied over the via hole 103. The layer 104 is in electrical contact with the electrically conductive core 102. The layer 104 has been applied to the coated lead over the via hole 103 by coating.

FIG. 1d illustrates 100d a coated lead with a via hole 103, a layer of PEDOT:PSS 104 over the via hole and an electrically conductive ring 105 located over the layer 104 and the via hole 103. The conductive ring has been crimped around the lead so as to make electrical contact with the layer 104. In this case, the conductive ring is of rhodium/platinum alloy (20% rhodium and 80% platinum, by mass), and has a thickness of 0.5 mm.

FIG. 2 illustrates schematically a process according to one embodiment for contacting a coated lead. FIG. 2 illustrates how the items 100a, 100b, 100c & 100d are related by process steps. In step a), a coated lead 100a is provided. In step b) 201, a via hole is provided in the insulating coating of the coated lead 100a to obtain a coated lead with a via hole 100b. In step c) 202, a layer is applied to the coated lead over the via hole to obtain a coated lead with a via hole and a layer over the via hole 100c. In step d) 203, an electrically conductive ring is crimped over the via hole and the layer to obtain the electrically contacted lead 100d.

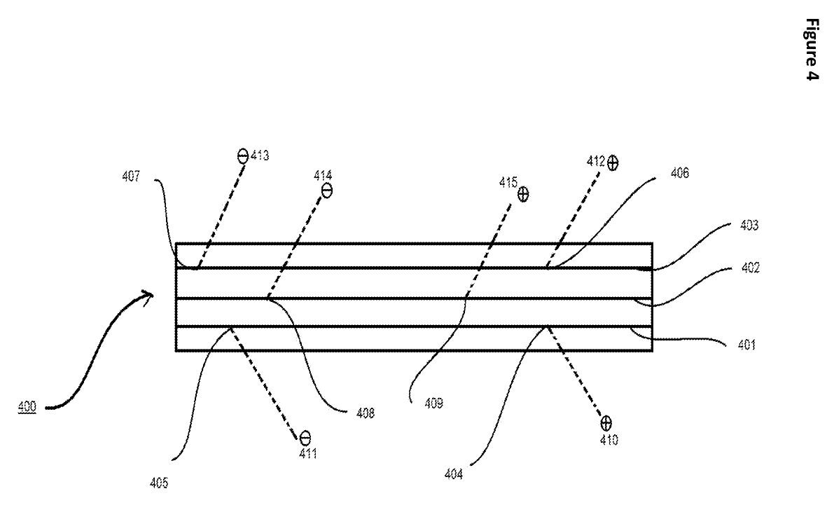

FIG. 3 illustrates schematically a pacemaker 50 with a pulse generator 70, and a lead 140 comprising an electrode 60. The lead 140 connects the pulse generator 70 and the heart tissue via the electrode 60. The lead 140 has been electrically connected to the pulse generator 70 and the electrode 60 by a process according to one embodiment.

FIG. 4 illustrates schematically a lead 400, comprising 3 electrically conductive cores 401, 402 & 403. Electrically conductive core 401 is electrically connected according to one embodiment at points 404 and 405 to allow an electrical circuit between notional terminals 410 and 411 respectively. Similarly, electrically conductive core 402 is electrically connected according to one embodiment at points 408 and 409 to allow an electrical circuit between notional terminals 414 and 415 respectively. Similarly, electrically conductive core 403 is electrically connected according to one embodiment at points 406 and 407 to allow an electrical circuit between notional terminals 412 and 413 respectively.

### Test Methods

### Fatigue Test

Fatigue resistance is measured according to the test described in “prEN 45502 Parts 2 & 3 CEN/CENELEC, Active Implantable Medical Devices—Brady and Tachy Lead Tests Draft/Standard”.

### EXAMPLES

### Example 1 (Inventive)—Contacting of a Lead according to one Embodiment

Electrical contacts were made to 15 cm long leads having a 0.2 mm diameter core of rhodium/platinum alloy (20% rhodium, 80% platinum, by mass) and a 20 μm coating of PTFE. 1 cm from each end of the lead, an electrical contact was made as follows: First, a via hole was made through the PTFE coating by laser ablation using a Varydisk laser available from Dausinger+Giesen GmbH. The via hole had a circular cross section of 60 μm diameter. Second, a layer of PEDOT:PSS was coated by spraying over the via hole in a cylindrical layer having a thickness of 30 μm and a cylinder length of 200 μm. The lead was heated to 100° C. for 5 hours to dry the PEDOT:PSS layer. Third, a cylindrical ring of rhodium/platinum alloy (20% rhodium, 80% platinum, by mass) with outer diameter of 350 μm, an inner diameter of 260 μm and a cylinder length of 500 μm, available from Heraeus Deutschland GmbH & Co. KG, was threaded over the lead, positioned so as to cover the via hole with applied PEDOT:PSS layer and crimped tight onto the lead using a multi-jaw chuck.

### Example 2a (Comparative)—Contacting of a Lead by Soldering

The lead was provided as in example 1 and electrical contact was made 1 cm from each end of each lead as follows: First, a via hole was made through the PTFE coating by laser ablation using a Varydisk laser available from Dausinger+Giesen GmbH. The via hole had a circular cross section of 60 μm diameter. Second, a cylindrical ring as in example 1 was threaded over the lead, positioned so as to cover the via hole and a contact was made between the ring and the core of the lead by soldering with gold at 700° C.

### Example 2b (Comparative)—Contacting of a Lead by Soldering

The lead was provided as in example 1 and electrical contact was made 1 cm from each end of each lead as follows: First, a via hole was made through the PTFE coating by laser ablation using a Varydisk laser available from Dausinger+Giesen GmbH. The via hole had a circular cross section of 60 μm diameter. Second, a cylindrical ring as in example 1, but with a 60 μm via hole was provided. The ring was threaded over the lead and positioned such that the via in the ring was over the via in the coating of the lead. Contact was made between the ring and the core of the lead by soldering through the two superimposed via holes with gold at 700° C.

### Example 3 (Comparative)—Contacting of a Lead by Crimping

A lead was provided as in example 1 and electrical contact was made 1 cm from each end of each lead as follows: First, a cylindrical ring as in example 1 was threaded over the lead and positioned 1 cm from the end of the lead. Second, the ring was crimped using a multi-jaw chuck to contact the ring with the core of the lead.

Properties of the Contacted Leads

For each of the examples (1, 2a, 2b & 3), 5 leads were contacted as described. Each contacted lead was tested for fatigue resistance. The success rate of contacting the leads and the results for fatigue resistance are illustrated in table 1.

TABLE 2

| Example | Successful contacting ? | Fatigue resistance |
|---|---|---|
| 1 | Yes, in all 5 samples | ++ |
| 2a | 2 leads contacted, 3 failed | −− |
| 2b | 3 leads contacted, 2 failed | −− |
| 3 | 4 leads contacted, 1 failed | −− |

++ Very good fatigue resistance (no failures after 400,000,000 cycles),
−− very poor fatigue resistance (failed after an average of less than 300,000 cycles)

What is claimed is:

1. A method for electrically contacting a coated lead comprising:
a. providing a coated lead comprising an electrically conductive core and an electrically insulating coating;
b. providing a via hole in the electrically insulating coating in order to expose a section of the electrically conductive core;
c. applying a first electrically conductive material to the coated lead such that it contacts at least a part of the exposed section of the electrically conductive core via the via hole; and
d. applying a second electrically conductive material to the coated lead such that it contacts at least a part of the first conductive material.

2. The method of claim 1, wherein the first electrically conductive material comprises a polymer.

3. The method of claim 1, wherein the first electrically conductive material is one or more selected from the group consisting of: an electrically conductive polymer, a composite comprising an electrically non-conductive polymer and an electrically conductive particle, and a composite comprising an electrically conductive polymer and an electrically conductive particle.

4. The method of claim 1, wherein the first electrically conductive material comprises a polymer having two or more conjugated double bonds.

5. The method of claim 1, wherein the first electrically conductive material is applied as a layer with thickness less than about 0.3 mm.

6. The method of claim 1, wherein the first electrically conductive material is applied by one or more selected from the group consisting of: dipping, printing, spraying, injecting, painting, dropping, stamping, spilling and laying.

7. The method of claim 1, wherein the second electrically conductive material is applied by solid deformation.

8. The method of claim 1, wherein the lead has a diameter less than about 2 mm.

9. The method of claim 1, wherein the lead comprises 2 or more electrically conductive cores.

10. The method of claim 9, wherein 2 or more of the electrically conductive cores are electrically insulated from each other by an insulated material.

11. The method of claim 1, wherein the via hole exposes a single conductive core.

12. The method of claim 1, wherein the provision of the via hole in step b. is performed with a laser.

13. An electrically contacted lead obtainable by a method of claim 1.

14. An electrically contacted lead comprising:
a. an electrically conductive core
b. an electrically insulating coating which coats the electrically conductive core;
c. a via hole in the electrically insulating coating which exposes a section of the electrically conductive core;
d. a first electrically conductive material which contacts at least a part of the exposed section of the electrically conductive core via the via hole; and
e. a further electrically conductive material which contacts at least a part of the first conductive material.

15. A medical device comprising a contacted lead according to claim 14.
